(19) United States
(12) Patent Application Publication
(10) Pub. No.: US 2009/0305390 A1
(43) Pub. Date: Dec. 10, 2009

(54) METHOD AND INSTALLATION FOR PROCESSING WASTE AND PRODUCING METHANE

(75) Inventors: Jesus CACHO, Mantes La Jolie (FR); Olivier CAUDART, Courtry (FR); Thomas LAGIER, Blaru (FR); Pascal PESLERBE, Lamorlaye (FR); Vassilia VIGNERON, Les Mureaux (FR)

Correspondence Address: FLEIT GIBBONS GUTMAN BONGINI & BIANCO P.L., ONE BOCA COMMERCE CENTER, 551 NORTHWEST 77TH STREET, SUITE 111, BOCA RATON, FL, 33487, US

(73) Assignee: Veolia Proprete, Nanterre (FR)

(21) Appl. No.: 12/182,413

(22) Filed: Jul. 30, 2008

(30) Foreign Application Priority Data
Jun. 5, 2008 (FR) 0853724

(51) Int. Cl. B09B 3/00; C12M 1/18; C02F 11/04

(52) U.S. Cl. 435/262; 210/603; 435/290.4

(57) ABSTRACT

A method is provided for processing waste and producing methane, in which a chamber is filled with waste, in which it undergoes anaerobic degradation. According to the method, a large chamber and a small chamber are filled respectively with slightly organic waste and highly organic waste, and a liquid fraction generated by the degradation of the waste in the large chamber is introduced into the small chamber. Also provided is an apparatus adapted to implement such a method.

### CROSS-REFERENCE TO RELATED APPLICATIONS

This application is based upon and claims priority from French Patent Application No. 08 53724, filed Jun. 5, 2008, the entire disclosure of which is herein incorporated by reference.

### FIELD OF THE INVENTION

The present invention concerns, in general terms, the field of the processing and recycling of waste.

More precisely, the invention concerns, according to a first of its aspects, a method of processing waste and producing methane. This method comprises an initial operation of filling at least one chamber with the waste, and an operation of anaerobic degradation of the waste in at least one filled chamber, during which methane is produced.

### BACKGROUND OF THE INVENTION

This type of method is known through the example of document FR 2 812 570.

The method described in the document FR 2 812 570 comprises several steps, including in particular preparation of the waste by shredding, the filling of the waste into an anaerobic cell, and control of moisture and temperature. The cell is then reused after reopening and the waste undergoes post-treatment in order to be recycled. A single type of reactor, here the anaerobic cell, is used. This method makes it possible to process only waste of similar quality, here municipal solid waste (MSW) and commercial and industrial waste (C&I).

The storage of waste such as in a landfill is known. The landfill accepts all waste as it is, in a large volume in which its degradation takes place naturally. However, this waste processing method has the drawback of taking a long time, in general at least a period of thirty years.

Methods of anaerobic digestion of waste are also known, in which the degradation of the waste is activated, in particular by the addition of micro-organisms, so that it takes place more rapidly than in a landfill. However, this waste processing method does not make it possible to process all waste.

### SUMMARY OF THE INVENTION

In this context, the aim of the present invention is to propose a method free from at least one of the limitations mentioned above and in particular a method allowing a more effective production of methane.

For this purpose, the method of the invention, moreover in accordance with the generic definition that the above preamble gives of it, is essentially characterised in that:
during the initial filling operation, at least one large chamber and at least one small chamber are filled respectively with slightly organic waste and highly organic waste, a large chamber having a volume capacity at least twenty five (25) times greater than the volume capacity of a small chamber,
the slightly organic waste, such as municipal solid waste and/or commercial and industrial waste, has a lower organic load than the highly organic waste, which is degradable in less than three years, the highly organic waste preferably being solid, and comprising for example at least one of the types of waste chosen from sludge from a waste water treatment plant, a fermentable fraction of municipal solid waste, organic waste processing residues, food waste, garden waste, waste from the agri-food industry and agricultural residues, and
during the degradation operation, a liquid fraction generated in at least one large chamber is introduced into at least one small chamber.

The invention makes it possible to degrade the waste organic matter in an accelerated fashion, whilst producing methane in order to recover it for energy. For this purpose, the invention relies on a synergy between bioreactors containing different qualities of waste.

Bioreactor means a chamber dedicated to the degradation of waste.

Degradation of waste means a decomposition of the organic matter that it contains, due to a biological action in particular of micro-organisms, enzymes and/or fungi. According to the definition of the invention, terms equivalent to the term “degradation” may be “digestion” or “fermentation” or “biodegradation”.

A degraded waste, in the present invention, means a waste where at least 60% of the initial biogas potential has been obtained, that is to say a waste that has produced 60% of the biogas that can be obtained under optimum laboratory conditions, or means a waste where at least 60% of the initial solubilisable carbon has been obtained, that is to say a waste that has lost at least 60% of the carbon that can be lost under optimum laboratory conditions.

In the invention, the degradation of the waste takes place in the absence of oxygen. This is therefore an anaerobic degradation.

The method of the invention makes it possible in particular to be more flexible than a simple above-ground anaerobic digestion method and more rapid than conventional landfilling.

The method of the invention has the advantage of obtaining acceleration of the degradation of the waste and consequently acceleration of the production of biogas, and therefore of recovery methane.

According to a preferential version of the invention, a liquid fraction generated in at least one small chamber is introduced into at least one large chamber.

Advantageously, the initial filling operation is not preceded by any mechanical treatment of the waste.

Conventionally, some chambers are reusable, or in other words reversible, that is to say, in these chambers, an excavation of the waste takes place, which makes it possible to reuse them for degrading new waste therein.

The processing periods in these reversible chambers depend on the quality of the incoming waste which, in the invention, is chosen so that its degradation in a small chamber according to the invention does not exceed a period of three years.

Other chambers are said to be “irreversible” or “fixed”. Once such chambers are filled with waste, they are not reopened in order to extract the degraded waste therefrom.

In the invention, preferably, at least one large chamber is irreversible while at least one small chamber is reversible.

A large chamber is for example a cell of a landfill, and more particularly a cell of a non-hazardous landfill.

Thus the invention proposes a coupling between a chamber, such as an anaerobic digestion reactor vessel, and a non-hazardous landfill cell, this coupling making it possible in particular to process waste that could not be processed directly in a conventional anaerobic digestion vessel.

The method according to the invention may also comprise a final operation of excavating the degraded waste from at least one small chamber.

This excavation of the waste if necessary makes it possible to establish one or more operations of reuse the material corresponding to the degraded waste, such as use as a solid recovered fuel, agricultural reuse, or recycling, and also makes it possible to subscribe to a policy of sustainable development by reusing the chamber for the processing of new waste.

One of the aims of the invention is to increase the water content of the waste and to seed it, in order to optimise its degradation.

For this purpose, the chambers may be seeded by liquid fractions, or leachates, which they generate, and/or by the liquid fractions coming from at least one other chamber.

In addition to these seedings by means of liquid fractions, the method of the invention may comprise, preferably during the initial filling operation, a seeding operation consisting of introducing, in at least one small chamber and/or at least one large chamber, micro-organisms, fungi and/or enzymes so that they participate in the degradation of the waste.

In a particular embodiment of the invention, at least one liquid-medium chamber is filled, continuously or discontinuously, with highly organic liquid waste, such as industrial liquid effluent issuing from the agri-food industry, the liquid waste undergoing, in at least one liquid-medium chamber, the anaerobic degradation operation, a large chamber having a volume capacity at least 25 times greater than the capacity volume of a liquid-medium chamber; and the liquid fraction generated in at least one large chamber and/or the liquid fraction generated in at least one small chamber is introduced into at least one liquid-medium chamber.

Advantageously, a liquid fraction generated in at least one liquid-medium chamber by the anaerobic degradation operation is introduced into at least one large chamber and/or into at least one small chamber.

A liquid-medium chamber according to the invention is preferably above ground.

In the invention, the chambers are fluid-tight. In particular, oxygen does not enter therein.

The method according to the invention may also comprise a preprocessing operation consisting of at least one of the liquid fractions generated respectively in at least one large chamber, in at least one small chamber and in at least one liquid-medium chamber being processed before introduced into any one of these chambers, in particular by heat treatment, by nitrification, by the addition of buffer, by the addition of micro-organisms, by the addition of enzymes and/or by the addition of fungi.

The invention concerns, according to a second of its aspects, an installation for processing waste and producing methane, adapted to implement the method according to the invention. This installation comprises:
at least one large chamber adapted to receive slightly organic waste, such as a cell of a non-hazardous landfill, and at least one small chamber adapted to receive highly organic waste, a large chamber having a volume capacity at least 25 times greater than the capacity volume of a small chamber, and the slightly organic waste having a lower organic load than the highly organic waste, which is degradable in less than three years,
means of introducing a liquid fraction issuing from at least one large chamber into at least one small chamber, and preferably also
means of introducing a liquid fraction issuing from at least one small chamber into at least one large chamber.

According to a particular embodiment of the invention, at least one small chamber is adapted to receive highly organic solid waste, and the installation also comprises:
at least one liquid-medium chamber adapted to receive highly organic liquid waste, and a large chamber having a volume capacity at least 25 times greater than the capacity volume of a liquid-medium chamber, and
means of introducing a liquid fraction issuing from at least one large chamber and/or a liquid fraction issuing from at least one small chamber into at least one liquid-medium chamber.

Advantageously, the installation according to the invention also comprises means of introducing a liquid fraction from at least one liquid-medium chamber to at least one large chamber and/or to at least one small chamber.

### BRIEF DESCRIPTION OF THE DRAWING

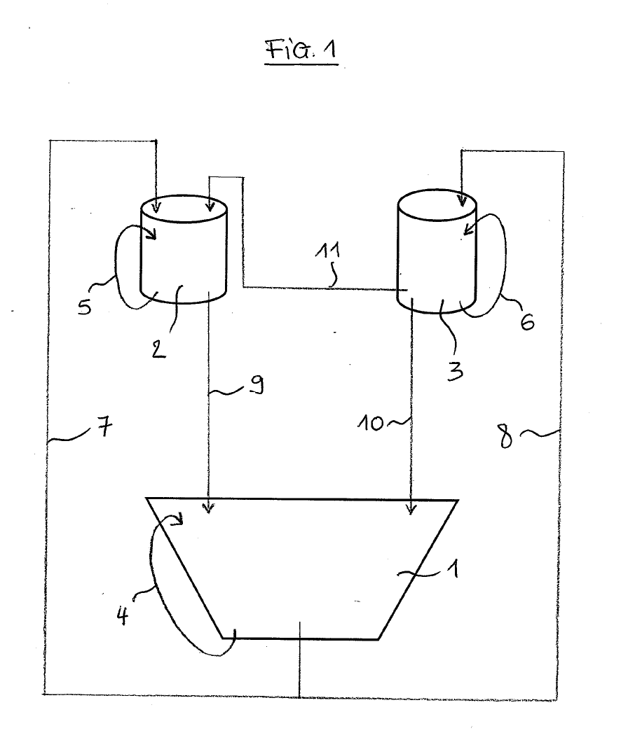

Other characteristics and advantages of the invention will emerge clearly from the detailed description that is given of it below, by way of indication and in no way limitatively, with reference to the accompanying FIG. 1 showing schematically an installation according to an embodiment of the present invention implementing a method according to an embodiment of the present invention.

### DETAILED DESCRIPTION OF PREFERRED EMBODIMENTS

Preferred embodiments of the present invention will be described in detail hereinbelow with reference to the attached drawings.

The installation shown in FIG. 1 has undergone an initial operation of filling several chambers 1, 2, 3 with waste that has not previously undergone mechanical processing.

This is because, unlike known conventional methods, in the invention, the waste can be directly put in place with any prior selection that takes place solely on the origin of the waste, but without mechanical processing.

The filling is accompanied or not by seeding.

Seeding means an addition of micro-organisms and/or other molecules or organisms that are able to accelerate the degradation of the waste.

A characterisation of the waste can be carried out also during this filling operation. It is possible in particular to put in place an identification of the nature of the waste, a recording of the moisture content and/or the proportion of volatile solids, and/or an evaluation of the potential for producing methane. For example, the waste includes green waste, fruits, vegetables, meat, wood such as pallets, plastics material, paper, cardboard, sludge and/or textiles.

Once the chambers 1, 2, 3 are filled, the waste undergoes therein degradation under anaerobic conditions, that is to say without oxygen, and at a temperature preferably between 30° and 60° C., and most preferably at a temperature of 35° C.

The rate of degradation of the waste is increased by means of the injection of liquid fractions coming from the three types 1, 2, 3 of chamber. Optimisation of the production of methane is thus obtained.

In FIG. 1, the installation includes a large chamber 1, a small chamber 2 and a liquid-medium chamber 3.

The large chamber 1 is a permanent installation such as a non-hazardous landfill of the bioreactor type. It is placed in the ground.

The small chamber 2, above the ground, is an anaerobic digestion vessel, also referred to as a digester. According to the invention, a small chamber may otherwise be a sealed cell in the ground.

The liquid-medium chamber 3 is an above-ground reaction vessel.

The large chamber 1 has a volume capacity at least 25 times greater than the capacity volume of the small chamber 2, as well as than the capacity volume of the liquid-medium chamber 3.

For example, the small chamber 2 and/or the liquid-medium chamber 3 have a volume capacity of between 500 and 3000 cubic metres (m³), preferably between 1000 and 2000 m³.

The large chamber 1 has for example a volume capacity of between 80,000 m³ and 4,000,000 m³.

The method and installation according to the invention make it possible to process waste of diverse origins.

In the large chamber 1, the waste normally received at a non-hazardous landfill is initially introduced and then degraded. This waste is in particular municipal solid waste (MSW) and/or commercial and industrial waste (C&I).

In the small chamber 2, waste with a high organic compound content is introduced and degraded, such as sludge, the fermentable fraction of municipal solid waste, organic residues of waste processing, or any other waste containing a high organic fraction including in particular food waste, fermentable waste and garden waste.

The waste initially introduced into the chamber 2 is preferably devoid of material such as wood, textile, paper and cardboard, or contains only a small quantity thereof.

The waste in the chamber 2 is degradable in less than three years, or preferably in less than one year.

Finally, liquid waste having a high organic fraction, coming for example from industry, in particular the agri-food industry, is introduced and degraded in the liquid-medium chamber 3.

This chamber 3 is supplied, continuously or discontinuously, with liquid waste. When the liquid emerging from the chamber 3, after degradation of the waste, achieves the discharge standards imposed by current legislation, it can be evacuated in order to be replaced by an equivalent volume of new liquid waste. The residence time for the waste in the chamber 3 depends on its quality on entry.

The chambers 1, 2 and 3 are thus filled with waste each release of methane, which can then be collected and recovered.

The advantage of the waste landfilling, such as in the chamber 1, is to accept all the waste as it is, without preprocessing, in a large volume in which the degradation takes place naturally. However, this method of processing waste has the drawback of taking a long time.

The advantage of the anaerobic digestion of waste, such as in the chamber 2, is mainly to activate the degradation of this waste so that it takes place more rapidly than in landfills. However, this waste processing method does not make it possible to process all the waste.

The method of the invention is more effective than the known methods in that it offers synergy between the landfilling of waste, in particular in the chamber 1, and an anaerobic digestion of the waste, in particular in the chamber 2.

In addition, the invention proposes an advantageous use of the liquid fractions issuing from the various chambers 1, 2, 3.

This is because a liquid fraction issuing from a chamber is injected into another chamber or into this same chamber. These injections of liquid fraction consequently increase the water content of the waste in the chambers 1, 2, 3 and allow a circulation of organic matter and/or a seeding of the waste.

The various injection streams of these liquid fractions or leachates are as follows:
streams 4, 5 and 6: injection of the liquid fraction issuing from a chamber 1, 2 or 3 into this same chamber 1, 2 or 3;
streams 7 and 8: injection of the liquid fraction issuing from the large chamber 1 into the small chamber 2 and/or into the liquid-medium chamber 3; these streams 7, 8 optimise not only the use of the leachates, generated by the large chamber 1, by increasing the water content of the waste processed in the small chamber 2 and/or in the liquid-medium chamber 3, but also the degradation of the organic carbon dissolved in these leachates;
stream 9: injection of the liquid fraction issuing from the small chamber 2 into the large chamber 1;
streams 10 and 11: injection of the liquid fraction issuing from the liquid-medium chamber 3 into the large chamber 1 and into the small chamber 2.

The injection rate of the leachate of the stream 7 can be varied in order to obtain separation of the acidogenic and methanogenic phases in the small chamber 2. In this case, the change of the organic matter of the waste processed in the chamber 2 into the liquid fraction discharged from the chamber 2 is facilitated. This fraction, collected and analysed, can then be injected into the large chamber 1 so that methane is produced therein.

The rate of injection of a leachate into a chamber can otherwise be optimised so that the waste situated therein has the time to be degraded as far as the methane production stage. In this case, the streams 7 to 11 are optimised for the seeding with active micro-organisms of the waste situated in the chamber where the stream arrives.

A leachate can be subjected to various analyses on discharge from the chamber before being injected into a chamber, in order to know the physical and chemical characteristics thereof, such as pH and temperature.

As a consequence or not of the results of these analyses, a leachate issuing from a chamber can be processed before being introduced into the same or another chamber. This preprocessing may for example be a settling, a nitrification, an oxidation, an addition of buffer, an addition of reagent, a heating or a biological seeding, in particular with bacteria, viruses and/or fungi.

The installation according to the invention may for example include at least one vessel for heating leachate to a temperature of 35° C. so that the micro-organisms that it contains are under optimum conditions for degrading the organic matter of the waste to which this previously heated leachate is added.

It is also possible to fit a temperature sensor, in a small chamber in particular, in order to know the temperature within the waste and to be able to inject, after heating where necessary, a liquid fraction, issuing in particular from a large chamber, the temperature of which allows degradation of the waste.

At the discharge from a large chamber, it is checked that, preferably, the liquid fraction has a pH of at least 6.8.

Once the waste is degraded in the small chamber 2, a ventilation step is carried out before opening the chamber 2 in order to excavate the waste therefrom, so as to terminate and/or stop the degradation of the waste.

The excavated waste can then be shredded and composted before drying. Some of this waste thus processed after excavation can be used as organic fertiliser, and/or as solid recovered fuel, and another part can be buried in a class 2 and/or class 3 landfill.

The invention can involve several chambers of each type of chamber 1, 2 or 3.

By way of example, it is possible to use thirteen small chambers of the same type as the chamber 2 and in the ground, and to fill one of these thirteen small chambers per month so that there is always a chamber from which the treated waste is excavated in order to be recycled. The liquid fractions issuing from the thirteen small chambers are injected into a large chamber of the same type as the chamber 1, that is to say in a non-hazardous landfill cell managed in bioreactor mode. The liquid fraction issuing from this cell is also injected into the small chambers.

The invention also has the following advantages compared with existing methods:
not requiring pretreatment of the waste before filling a chamber;
flexibility with regard to anaerobic degradation, according to the waste and the metabolic pathway put in place in the reversible bioreactor (acidogenic phase only or combination of the two acidogenic and methanogenic phases), which is possible by virtue of the combination of a small reversible bioreactor and an irreversible bioreactor with a size at least 25 times greater; and
management of the waste and their by-products in the same place, thus avoiding having recourse to transport, and therefore affording a saving in time and a limitation to the pollution during transport, in particular by the release of greenhouse gas.

While there has been illustrated and described what are presently considered to be the preferred embodiments of the present invention, it will be understood by those skilled in the art that various other modifications may be made, and equivalents may be substituted, without departing from the true scope of the present invention. Additionally, many modifications may be made to adapt a particular situation to the teachings of the present invention without departing from the central inventive concept described herein. Furthermore, an embodiment of the present invention may not include all of the features described above. Therefore, it is intended that the present invention not be limited to the particular embodiments disclosed, but that the invention include all embodiments falling within the scope of the appended claims.

1. Method of processing waste and producing methane, the method comprising:
performing an initial operation of filling at least one chamber with the waste and
performing an operation of anaerobic degradation of the waste in at least one filled chamber, during which methane is produced,
wherein during the initial filling operation, at least one large chamber and at least one small chamber are filled respectively with slightly organic waste and highly organic waste, the at least one large chamber having a volume at least 25 times greater than a volume of the at least one small chamber,
the slightly organic waste has a lower organic load than the highly organic waste, which is degradable in less than three years, and
during the anaerobic degradation operation, a liquid fraction generated in the at least one large chamber is introduced into the at least one small chamber.

2. The method according to claim 1, wherein a liquid fraction generated in the at least one small chamber is introduced into the at least one large chamber.

3. The method according to claim 1, wherein the initial operation is not preceded by any mechanical treatment of the waste.

4. The method according to claim 1, further comprising performing a final operation of excavating the degraded waste from the at least one small chamber.

5. The method according to claim 1, wherein the initial operation comprises performing a seeding operation comprising introducing, into the at least one small chamber and/or the at least one large chamber, at least one of micro-organisms, fungi and enzymes that participate in the anaerobic degradation of the waste.

6. The method according to claim 1, wherein the large chamber is a cell of a non-hazardous landfill.

7. The method according to claim 1, further comprising:
filling at least one liquid-medium chamber, continuously or discontinuously, with highly organic liquid waste, the liquid waste undergoing, in the at least one liquid-medium chamber, the anaerobic degradation operation, the at least one large chamber having a volume at least 25 times greater than a volume of the at least one liquid-medium chamber; and
introducing the liquid fraction generated in the at least one large chamber and/or the liquid fraction generated in the at least one small chamber into the at least one liquid-medium chamber.

8. The method according to claim 7, further comprising introducing a liquid fraction generated in the at least one liquid-medium chamber by the anaerobic degradation operation into the at least one large chamber and/or into the at least one small chamber.

9. The method according to claim 1, further comprising performing a pretreatment operation in which at least one of the liquid fractions generated respectively in the at least one large chamber, in the at least one small chamber and in the at least one liquid-medium chamber is treated before being introduced into any one of the chambers.

10. An apparatus for processing waste and producing methane, the apparatus comprising:
at least one large chamber adapted to receive slightly organic waste;
at least one small chamber adapted to receive highly organic waste, the at least one large chamber having a volume at least 25 times greater than a volume of the at least one small chamber, and the slightly organic waste having a lower organic load than the highly organic waste, which is degradable in less than three years; and
means for introducing a liquid fraction issuing from the at least one large chamber into the at least one small chamber.

11. The apparatus according to claim 10, further comprising means for introducing a liquid fraction issuing from the at least one small chamber into the at least one large chamber.

12. The apparatus according to claim 10,
wherein the at least one small chamber is adapted to receive highly organic solid waste, and
the apparatus further comprises:
at least one liquid-medium chamber adapted to receive highly organic liquid waste, the at least one large chamber having a volume at least 25 times greater than a volume of the at least one liquid-medium chamber; and
means for introducing a liquid fraction issuing from the at least one large chamber and/or a liquid fraction issuing from the at least one small chamber into the at least one liquid-medium chamber.

13. The apparatus according to claim 12, further comprising means for introducing a liquid fraction from the at least one liquid-medium chamber to the at least one large chamber and/or to the at least one small chamber.

14. The method according to claim 1, wherein the slightly organic waste is at least one of municipal solid waste and commercial and industrial waste.

15. The method according to claim 14, wherein the highly organic waste is solid and comprises at least one of sludge from a waste water treatment plant, a fermentable fraction of municipal solid waste, organic waste processing residues, food waste, garden waste, residues from the agri-food industry, and agricultural residues.

16. The method according to claim 7, wherein the highly organic liquid waste comprises industrial liquid effluent issuing from the agri-food industry.

17. The method according to claim 9, wherein in the pretreatment operation, the at least one of the liquid fractions is treated by at least one of heat treatment, nitrification, addition of buffer, addition of micro-organisms, addition of enzymes, and addition of fungi.

18. The apparatus according to claim 10, wherein the at least one large chamber comprises a cell of a non-hazardous landfill.
